(19) United States
(12) Patent Application Publication
(10) Pub. No.: US 2009/0130769 A1
(43) Pub. Date: May 21, 2009

(54) Novel Cross-Linkers For Obtaining Structure Information On Molecule Complexes

(75) Inventors: Luitzen de Jong, Monnickendam (NL); Piotr Thomasz Kasper, Amsterdam (NL); Jaap Willem Back, Vleuten (NL); Jan Herman van Maarseveen, Amersfoort (NL); Chris Gerdinus de Koster, Hoorn (NL)

Correspondence Address: FOLEY HOAG, LLP;PATENT GROUP, WORLD TRADE CENTER WEST, 155 SEAPORT BLVD, BOSTON, MA, 02110, US

(73) Assignee: Universiteit van Amsterdam, Amsterdam (NL)

(21) Appl. No.: 12/067,758

(22) PCT Filed: Sep. 21, 2005
(86) PCT No.: PCT/NL2005/000686

§ 371 Date: Oct. 2, 2008

(51) Int. Cl. G01N 33/68; C07D 403/12; C07D 403/06; C07K 1/107

(52) U.S. Cl. 436/86; 548/520; 530/345

(57) ABSTRACT

The present invention describes a novel cross-linker, a method for preparing one or more cross-linked biomolecules, biomolecular complexes of two or more biomolecules, a method for preparing cross-linked fragments from such cross-linked biomolecules and/or biomolecular complexes, a method for cleavage and reduction of such cross-linked biomolecules and/or biomolecular complexes, a method for identifying cross-links in such cross-linked biomolecules and/or biomolecular complexes, as well as a method for determining relative amounts of cross-links in a biomolecule or biomolecular complex in two or more samples.

The present invention relates to a novel cross-linker, a method for preparing one or more cross-linked biomolecules, biomolecular complexes of two or more biomolecules, a method for preparing cross-linked fragments from such cross-linked biomolecules and/or biomolecular complexes, a method for cleavage and reduction of such cross-linked biomolecules and/or biomolecular complexes, a method for identifying cross-links in such cross-linked biomolecules and/or biomolecular complexes, as well as a method for determining relative amounts of cross-links in a biomolecule or biomolecular complex in two or more samples.

Chemical cross-linking is used to identify nearest neighbors in protein complexes, while identifying cross-linked amino acids residues is a powerful method to validate models of the 3-D structure of proteins and protein complexes (S. S. Wong, Chemistry of Protein Conjugation and Crosslinking. CRC Press: Boca Raton, USA, 1991; G. T. Hermanson, Bioconjugate Techniques. Academic Press: San Diego, USA, 1996; J. W. Back, L. de Jong, A. O. Muijsers & C. G. de Koster, J. Mol. Biol. 2003, 331, 303; A. Sinz, J. Mass Spectrom. 2003, 38, 1225; N. Geisler, FEBS Lett. 1993, 323, 63; M. G. F. E. Cohen & M. J. Sternberg J. Mol. Biol. 1980, 138, 3). However, mapping of cross-links is challenging, hampering wide application of the technique.

To map experimentally introduced cross-links, the protein complex under study is usually subjected to enzymatic or chemical cleavage to generate peptides of a convenient size, followed by isolation and recognition of cross-linked peptides. Finally, structural elucidation of the cross-linked peptides is carried out to identify the amino acid residues involved in the cross-link. Several approaches have appeared in literature for isolation, recognition, and identification. Especially the introduction of sensitive mass spectrometers for peptide analysis has boosted new strategies for the analysis of cross-links (J. W. Back, A. F. Hartog, H. L. Dekker, A. O. Muijsers, L. J. de Koning & L. de Jong, J. Am. Soc. Mass Spectrom. 2001, 12, 222; J. W. Back, V. Noteboom, L. J. de Koning, A. O. Muijsers, T. K. Sixma, C. G. de Koster & L. de Jong, Anal. Chem. 2002, 74, 4417; X. Tang, G. R. Munske, W. F. Siems & J. E. Bruce, Anal. Chem. 2005, 77, 311; E. V. Petrotchenko, V. K. Olkhovik & C. H. Borchers, Mol. Cell. Proteom. 2005, 4, 1167; T. Taverner, N. E. Hall, R. A. J. O'Hair & R. J. Simpson; J. Biol. Chem. 2002, 277, 46487; D. R. Muller, P. Schindler, H. Towbin, U. Wirth, H. Voshol, S. Hoving & M. O. Steinmetz, Anal. Chem. 2001, 73, 1927). Nevertheless, mapping of cross-links remains a difficult task, and experimental verification of models of 3-D structures of proteins, or charting the dynamics of protein complexes by chemical cross-linking has still not found wide application, let alone genome-wide mapping of protein-protein interaction by cross-linking. A major limitation of current analytical strategies is that, once candidate cross-linked peptides have been detected, their structural elucidation is often hampered by the complexity or insufficient quality of tandem mass spectra derived from such species. This is especially the case with relatively large protein complexes, where several theoretical possibilities exist for combinations of peptides corresponding to the measured mass of a candidate cross-linked peptide. Cross-linkers with a cleavable spacer (E. V. Petrotchenko, V. K. Olkhovik & C. H. Borchers, Mol. Cell. Proteom. 2005, 4, 1167; K. L. Bennett, M. Kussmann, P. Bjork, M. Godzwon, M. Mikkelsen, P. Sorensen & P. Roepstorff, Protein Sci. 2000, 9, 1503-18; J. W. Back, M. A. Sanz, L. de Jong, L. J. de Koning, L. G. Nijtmans, C. G. de Koster, L. A. Grivell, H. van der Spek & A. O. Muijsers, Protein Sci. 2002, 11, 2471) can only partially remedy this limitation. Cleavage products can not easily be assigned to cross-linked peptides in complex peptide mixtures, preventing wide application of cleavable cross-linkers. Moreover, disulfide interchange reactions, premature cleavage or side reactions complicate the use of available cleavable cross-linkers.

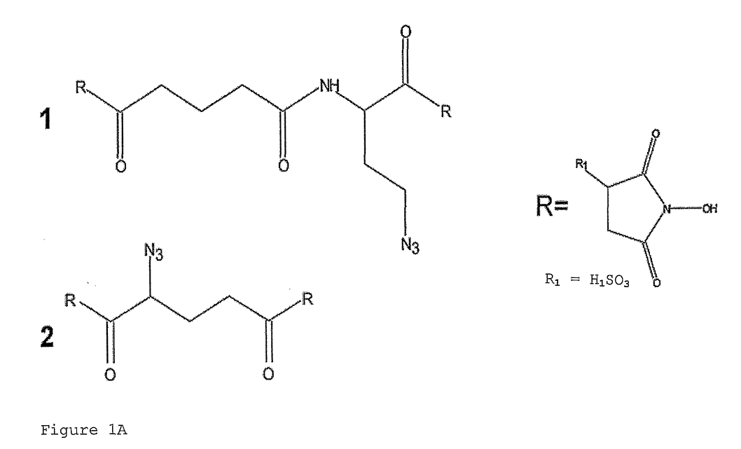

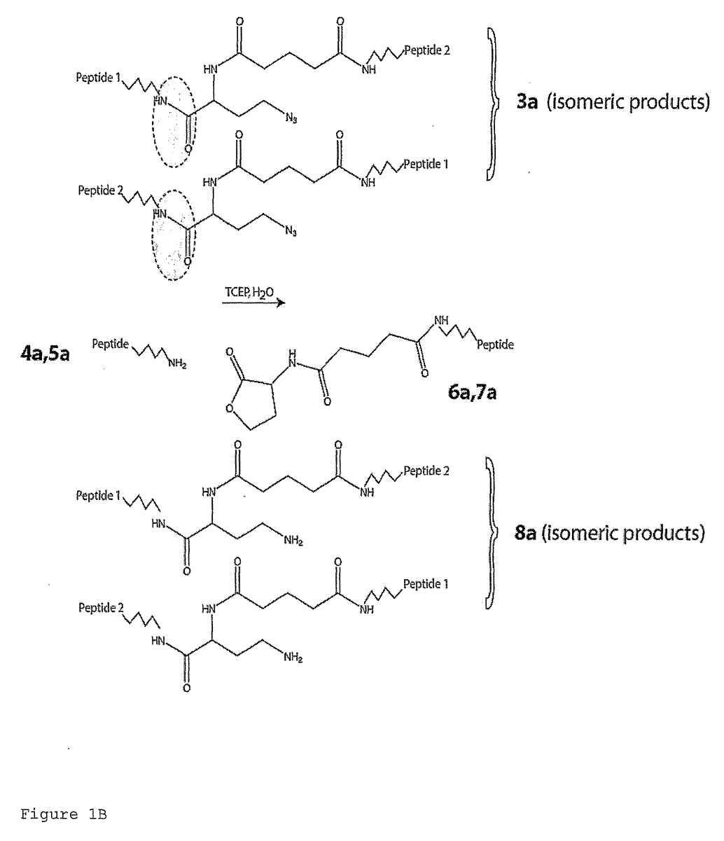

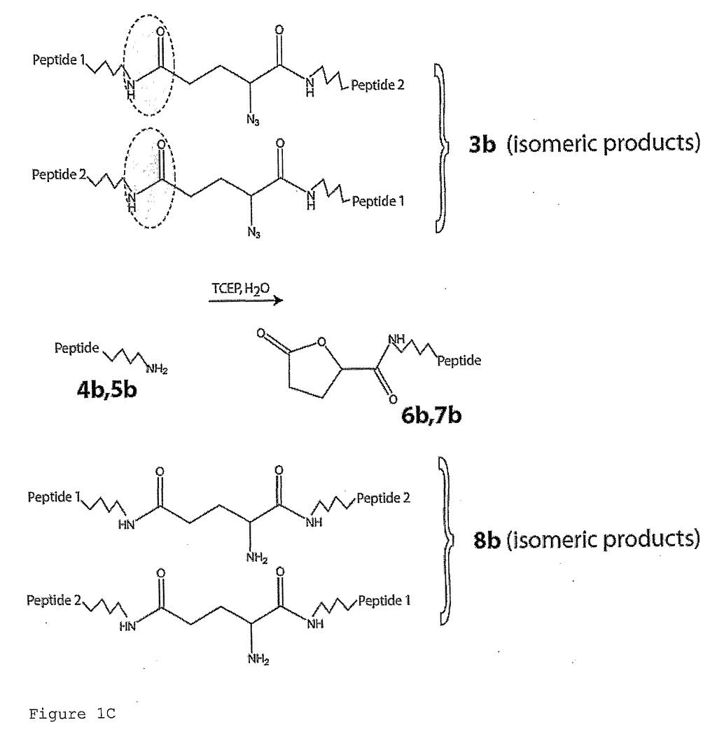

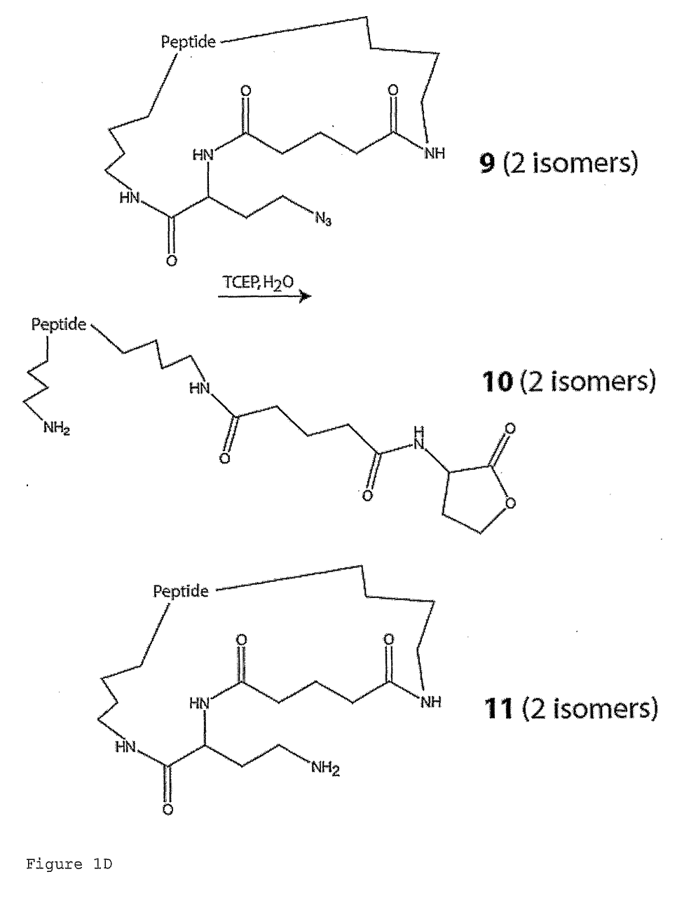

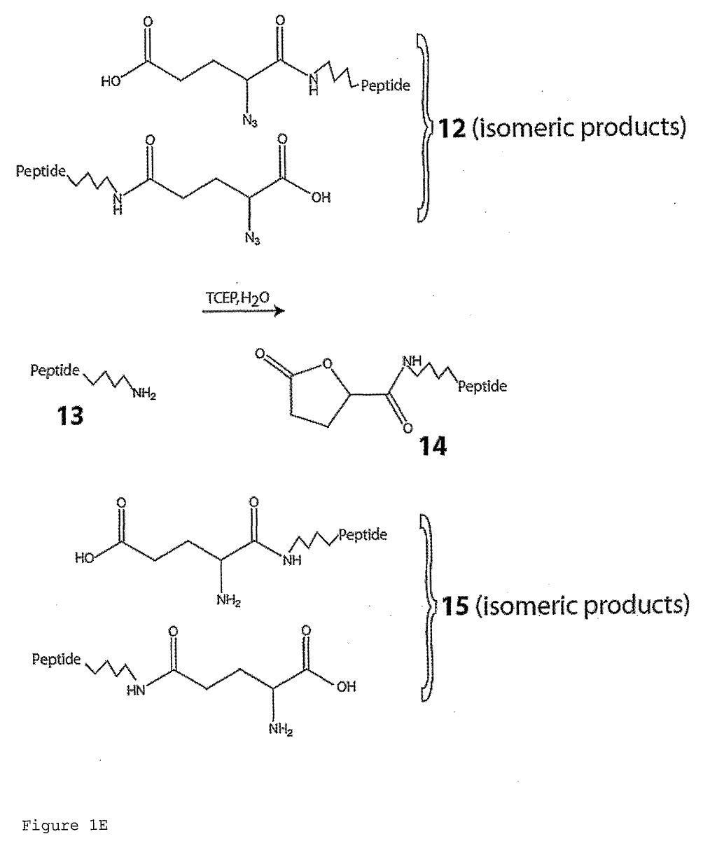

The present inventors now present a group of novel cross-linkers that make rapid and unambiguous identification of cross-linked sites in complexes of macromolecules by mass spectrometry possible. The present invention is herein exemplified by bis(sulfosuccinimidyl) 5-(3-azido-1-carboxypropylamino)-5-oxopentanoate (BACOX; see FIG. 1A, 1) and bis(succinimidyl) 2-azido-glutarate (NAG; see FIG. 1A, 2), but is not limited thereto. It was found that the precise positioning of an azide moiety in the spacers of the cross-linkers, e.g. BACOX or NAG, with respect to one of the two amide groups that are either present in the cross-linker or formed upon reaction with the molecules, e.g. proteins, provides these reagents with peculiar chemical properties after cross-linking, enabling such rapid and unambiguous identification. The present inventors demonstrate that the products formed by treatment of cross-linked biomolecules, such as peptides, with an azide-reducing agent such as a phosphine or thiol, can provide an easy clue towards the peptide identity of the cross-link. This feature enables easy assessment of cross-linked sites in e.g. protein complexes by mass spectrometry.

Thus, in a first aspect the present invention relates to a cross-linker having the following structure:

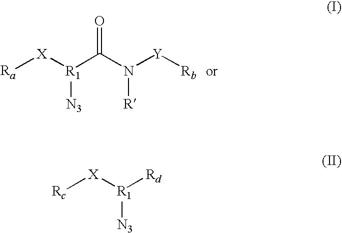

, wherein

Rₐ, R_{b}, R_{c} are functional end groups reactive with a functional cross-linking group on a biomolecule and Rₐ and R_{b} can be the same or different;

R_{d} is a functional end group reactive with an N-containing functional cross-linking group on a biomolecule as to form an amide bond;

X and Y are optional and can be any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether, polyacetal, polycarbonate, polysaccharide, polyamide, polypeptide, polyurethane and polyester moieties, and can be the same or different;

R₁ can be any (optionally substituted) moiety having 3 or 4 atoms as to provide a distance of 3 or 4 atoms between the azide group —N₃ and the carbonyl carbon atom of the amide bond; and

R′ can be hydrogen, or can be any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether, polyacetal, polycarbonate, polysaccharide, polyamide, polypeptide, polyurethane and polyester.

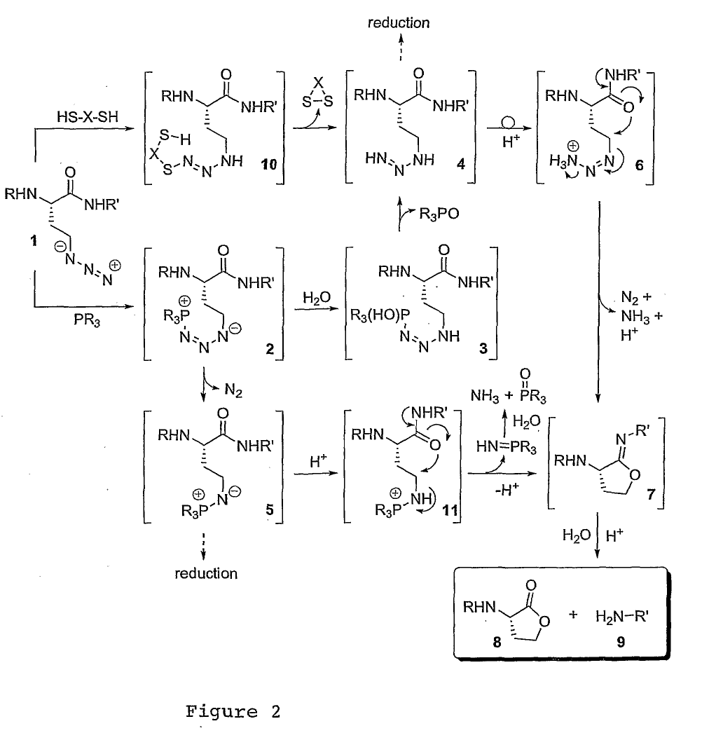

The basis for the design of the novel cross-linkers is the observation that in the presence of azide-reducing agents such as tris(2-carboxyethyl)phosphine (TCEP), or dithiothreitol, two competing reactions occur simultaneously in peptides containing e.g. the non-natural amino acid azidohomoalanine (J. W. Back, O. David, G. Kramer, G. Masson, P. T. Kasper, L. J. de Koning, L. de Jong, J. H. van Maarseveen & C. G. de Koster, Angew. Chem. 2005, in press). The two competing reactions result in a mixture of three products derived from the peptide. One reaction is hydrolysis of the peptide bond C-terminal to the azidohomoalanine residue. After hydrolysis the C-terminal peptide has a homoserine lactone residue at its C-terminus, while the N-terminal peptide is present as its free amine. The lactone is in a pH dependent equilibrium with homoserine. The other reaction is the reduction of the azide group in the azidohomoalanine residue in the intact peptide to an amine. A reaction scheme is depicted in FIG. 2.

All technical and scientific terms used herein have the same meaning as commonly understood by one of ordinary skill in the art to which this invention belongs. For clarification purposes only, some terms will be further illustrated below.

The term “cross-linker” is well known in the art and e.g. refers to a compound capable of establishing a covalent linkage between two functional cross-linking groups in a (bio)molecule or a complex of two or more (bio)molecules. Whether or not two functional groups in the (bio)molecule or (bio)molecular complex will cross-link depends on several factors, such as the distance between functional cross-linking groups within the biomolecule or complex of biomolecules, the length of the cross-linker and of course the conditions under which cross-linking is performed. The cross-linker consists of two functional end groups separated by means of a so-called spacer. The spacer, i.e. the portion of the cross-linker in between groups Rₐ and R_{b}, or R_{c}, and R_{d}, may be adjusted as to provide any desired length and any desired chemical composition.

The term “functional end groups” as used herein refers to end groups that are capable of reacting with functional cross-linking groups on biomolecules. Such functional end group may e.g. be α-haloacetyl compounds, N-maleimide derivatives, mercurials, aryl halides, aldehydes, ketones, isocyanates, isothiocyanates, imidoesters, acid halides, acid anhydride, N-hydroxysuccinimidyl and other activated esters, N-acetylimidazole, diazoacetate esters, diazoacetamides, carbodiimides, diazonium compounds, dicarbonyl reagents, epoxides, photoactivatable aryl azides, etcetera (S. S. Wong, Chemistry of Protein Conjugation and Crosslinking. CRC Press: Boca Raton, USA, 1991). Whereas several of these functional end groups are specific towards certain functional cross-linking groups in a biomolecule as exemplified below, some others, e.g. aryl azides may be rather unspecific, i.e., are able to react with many different functional cross-linking groups.

The term “functional cross-linking groups” as used herein refers to groups present on biomolecules that can react with functional end groups of the cross-linker according to the present invention to provide a cross-link. In the case of the molecules to be cross-linked being proteins or peptides, such functional cross-linking groups could e.g. be amine groups, e.g. present at the N-terminus of such proteins or peptides, or in the side chain of lysine residues, hydroxyl groups, e.g. present in the side chain of serine, threonine or tyrosine residues, carboxyl groups, e.g. present at the C-terminus of such proteins or peptides, or in the side chain of glutamic acid or aspartic acid residues, sulfhydryl groups, e.g. present in the side chain of cysteine residues, guanidinium groups, e.g. present in the side chain of arginine residues, imidazole groups, e.g. present in the side chain of histidine residues, etcetera. Similarly, certain groups present in DNA or RNA or other molecules to be cross-linked may be functional cross-linking groups, in particular towards functional end groups with low specificity, such as the said photoactivatable aryl azides.

The term “having 3 or 4 atoms as to provide a distance of 3 or 4 atoms between the azide group —N₃ and the carbonyl carbon atom of the amide bond” is used to indicate the spatial distance of the azide group to the carbonyl carbon atom of the amide bond to be cleaved. In the case of a peptide-like cross-linker, e.g. comprising a non-natural azide-functionalized amino acid such as azidohomoalanine, such amino acid may be located N-terminal to the amide bond to be cleaved. Non-limiting examples of such non-natural azide-functionalized amino acid are azidohomoalanine, azidonorvaline, and derivatives thereof (substituted azidohomoalanine or azidonorvaline. With the term “a distance of 3 or 4 atoms between the azide group —N₃ and the carbonyl carbon atom of the amide bond” is meant the distance between the nitrogen atom of the azide group α to R₁ and the carbonyl carbon atom. Effectively, this means that the nitrogen atom of the azide group a to R₁ is separated from the carbonyl carbon atom of the amide bond by 3 or 4 atoms.

In a preferred embodiment, the distance between the azide group —N₃ and the carbonyl carbon atom of the amide bond is 3 atoms, preferably 3 carbon atoms, as these are known to provide the correct geometry for formation of a five-membered ring, e.g. homoserine lactone formed in the cleavage reaction depicted in FIG. 2.

In the present invention, the amide bond to be cleaved may be present in the cross-linker itself (cross-linker I) or may be formed upon cross-linking of one of the functional end groups with a functional cross-linking group on a biomolecule to be cross-linked (cross-linker II). An amide bond is e.g. formed upon reaction of an acid group with an amine group, an N-hydroxysuccinimidyl or other activated ester with an amine group, an acid anhydride with an amine group or an acyl halide with an amine group.

Rₐ, R_{b}, R_{c}, are functional end groups that are reactive with a functional cross-linking group on a (bio)molecule and Rₐ and R_{b} can be the same or different. The analytical strategy described herein can be carried out with any type of functional end groups (Wong SS. Chemistry of Protein Conjugation and Crosslinking. CRC Press: Boca Raton, USA, 1991; Hermanson GT. Bioconjugate Techniques, Academic Press: San Diego, USA, 1996), e.g., (i) functional end groups directed to an amino group, such as imidoesters, aryl halides, acylating agents, aldehydes, ketones and others; (ii) functional end groups directed to a sulfhydryl group, such as maleimides, alkylating agents, haloacetyl derivatives, alkyl halides, s-triazines, aziridines and epoxides; (iii) functional end groups directed to a carboxyl group, such as diazo acetate esters, diazoacetamides and carbodiimides; (iv) functional end groups directed to a phenolate such as N-acetylimidazole and diazonium compounds, (v) functional end groups directed to an arginine such as 1,2 dicarbonyl reagents; (vi) photo-activatable end groups such as aryl azides that react indiscriminately with amino acid side chains in proteins or with purine, pyrimidine, or (deoxy)ribose residues in nucleic acids; and (vii) any combination of two different functional end groups mentioned in (i) through (vi), as long as the cross-linker comprises an azide group positioned 3 or 4 atoms from the carbon of an amide bond that can be cleaved. As such, the cross-linker according to the present invention may have two identical functional end groups Rₐ and R_{b} or R_{c}, and R_{d} or may comprise two different functional end groups.

R_{d} is a functional end group reactive with an N-containing functional cross-linking group on a biomolecule as to form an amide bond. In this embodiment of the present application, the amide bond is formed upon reaction of the functional end group with the functional cross-linking group. Thus, R_{d} should specifically be reactive with an N-containing functional cross-linking group.

The reactions underlying cross-linking with the cross-linkers according to the present invention are the same as for any cross-linker known in the art. E.g., a primary reaction of BACOX or NAG with an amine is followed either by reaction with a neighbouring amine in the protein, leading to a cross-link, or hydrolysis of the remaining activated ester, leading to a single modification without actual cross-linking.

X and Y are optional and are used to elongate the spacer. X and Y can be any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether, polyacetal, polycarbonate, polysaccharide, polyamide, polypeptide, polyurethane and polyester moieties. The spacer is defined as that part of the cross-linker connecting the two functional end groups. X and Y may be the same moiety or may be different. X and Y can have any length as to provide for cross-linkers having spacers with different lengths. Thus, cross-linking agents with an aptly positioned azide in the spacer can be provided with spacers of any length. For example, X and Y can be polyethylene glycol, ethylene glycol, etcetera.

R₁ can be any (optionally substituted) moiety having 3 or 4 carbon atoms as to space the azide group —N₃ 3 or 4 carbon atoms from the carbonyl carbon atom of the amide bond. Preferably, R₁ is an optionally substituted C3 or C4 compound.

R′ can be hydrogen, or can be any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether, polyacetal, polycarbonate, polysaccharide, polyamide, polypeptide, polyurethane and polyester.

In an embodiment, Rₐ, R_{b}, and R_{c}, can be the same or different and are chosen from the group, consisting of α-haloacetyl compounds, N-maleimide derivatives, mercurials, aryl halides, aldehydes, ketones, isocyanates, isothiocyanates, imidoesters, acid halides, acid anhydride, N-hydroxysuccinimidyl and other activated esters, N-acetylimidazole, diazoacetate esters, diazoacetamides, carbodiimides, diazonium compounds, dicarbonyl reagents, epoxides, and aryl azides. It is known in the art that such groups are particularly suited as functional end groups for cross-linking purposes.

In a preferred embodiment, R′ is hydrogen. It was found that such substitution on the nitrogen atom of the amide bond was suitable for the required cleavage of the amide bond induced by reducing agents like phosphines and thiols. It is conceivable that also modification of the amine nitrogen with any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether and polyester will give the required cleavage products with reducing agents, like phosphines or thiols.

In a further aspect, the present invention relates to the specific cross-linker bis(sulfosuccinimidyl) 5-(3-azido-1-carboxypropylamino)-5-oxopentanoate. It was found that this cross-linker was particularly suitable for the purposes of the present invention.

In yet a further aspect, the present invention relates to the specific cross-linker bis(succinimidyl) 2-azido-glutarate. It was found that also this particular cross-linker, was highly suitable for the purposes of the present invention.

As is well known in the art, elements can exist in both stable and unstable (radioactive) forms. Most elements of biological interest (including C, H, O, N, and S) have two or more stable isotopes, with the lightest of these present in much greater abundance than the others. Among stable isotopes the most useful as biological tracers are the heavy isotopes of carbon and nitrogen. These two elements are found in the earth, the atmosphere, and all living things. Each has a heavy isotope (¹³C and ¹⁵N) with a natural abundance of ˜1% (¹³C) or ˜0.4% (¹⁵N) and a light isotope (¹²C and ¹⁴N) that makes up all of the remainder, in the case of nitrogen, or virtually all in the case of carbon (carbon also has a radioactive isotope, ¹⁴C). Stable isotopes are often used for quantitative analysis using mass spectrometry. For a review on protein quantification by mass spectrometry, see. J. Listgarten & A. Emili, Mol. Cell. Proteom. 2005, 4, 419 or M. B. Goshe & R. D. Smith, Curr. Opin. Biotechnol. 2003, 14, 101.

In an embodiment, the cross-linker according to the present invention, and preferably the spacer thereof, further comprises one or more isotopes of an element. The skilled practitioner in the field of organic synthesis is well aware of methods suitable for the incorporation such heavy isotopes into an organic compound. It is preferred that such isotopes are heavy isotopes to replace the naturally most frequently occurring ‘light’ isotopes. Such cross-linker comprising e.g. a heavy isotope of an element can advantageously be used in combination with a chemically identical cross-linker comprising a light isotope at the same position. Such sets of comparable cross-linkers (previously described by e.g. D. R. Muller, P. Schindler, H. Towbin, U. Wirth, H. Voshol, S. Hoving, M. O. Steinmetz, Anal. Chem. 2001, 73, 1927, or C. J. Collins, B. Schilling, M. Young, G. Dollinger, R. K. Guy, Bioorg Med Chem Lett 2003, 13, 4023), one comprising one or more heavy isotopes in one or more positions and another comprising one or more light isotopes in the same position(s), can advantageously be employed for massaspectrometric analysis for determining relative or absolute amounts of biomolecules or biomolecular complexes. A useful application of such analysis would e.g. be an investigation in the change in pattern of protein-protein interactions in a cell over time following activation of a receptor and the concomitant signal transduction pathway. One could for example prepare two cell extracts: an extract at t=0 and an extract on t=x. One of the extracts could be cross-linked with the novel cross-linker according to the present invention comprising one or more ‘light’ isotopes, whereas the other extract could be cross-linked with the novel cross-linker according to the present invention comprising one or more ‘heavy’ isotopes. One such example can e.g. be wherein the ‘light’ isotope in the first cross-linker is ¹²C and in the second cross-linker e.g. 6 ¹²C atoms are replaced by ¹³C, such that the molar mass of the second cross-linker is increased by 6 Da compared to the first cross-linker. Stable isotopes are preferably to be incorporated in the spacer, since the functional end groups are generally removed during cross-linking. Therefore, the maximum molar mass difference between the first and second cross-linker is limited by the size and elemental composition of the spacer of the cross-linker. The molar mass difference between the first and second cross-linker is preferably at least 2 Da, more preferably at least 4 Da, yet more preferably at least 6 Da, most preferably at least 8 Da. Such molar mass difference gives sufficient peak resolution in most mass spectrometers to enable accurate determination of peak area ratios of signals from light and heavy istopically labelled compounds, respectively. The one or more heavy isotopes can be chosen from any heavy isotopes available for labelling of compounds. For illustration purposes, ¹H in the first cross-linker can e.g. be replaced by ²H in the second cross-linker, ¹²C in the first cross-linker can e.g. be replaced by ¹³C in the second cross-linker, etcetera. One skilled in the art will readily be capable of selecting suitable isotopes to incorporate in the cross-linker according to the present invention.

In a preferred embodiment, the one or more isotopes of an element are chosen from the group consisting of ¹³C, ¹⁵N and/or ¹⁸O, since the use of such isotopes gives no difference in retention time with reversed phase liquid chromatography, a separation technique often used in conjunction with mass spectrometry.

In yet a further embodiment, the present invention relates to a kit comprising at least a (first) cross-linker according to the present invention. Said kit may further comprise other components, preferably chosen from one or more of i) a second chemically identical cross-linker comprising isotopes, such that said second cross-linker differs in molar mass from the first cross-linker, preferably displaying a molar mass difference of 2 Da or more; ii) an azide-reducing agent; iii) a buffer with the optimal pH for the cross-linking reaction.

The cross-linker according to the present invention may be prepared by any method known in the art, and may for example be prepared using an azide-functionalized amino acid such as azidohomoalanine, azidonorvaline and derivatives thereof. However, the preparation method for the cross-linker is not limited thereto. A skilled practitioner in organic synthesis will readily be capable of preparing the cross-linker according to the present invention. For illustrative purposes only, the preparation methods of bis(sulfosuccinimidyl) 5-(3-azido-1-carboxypropylamino)-5-oxopentanoate and bis(succinimidyl) 2-azido-glutarate are e.g. disclosed in examples 1 and 2, respectively.

Preferably, the cross-linker as defined above is prepared using an azide-functionalized spacer, since the functional end groups are generally removed during cross-linking.

In a further aspect, the present invention relates to a method for preparing one or more cross-linked biomolecules, biomolecular complexes of two or more biomolecules or mixtures thereof, said method comprising the step of using the cross-linker as defined above. As will be further discussed below, such cross-linked biomolecules or biomolecular complexes or mixtures thereof can easily be used for analysis aimed at identification of cross-linked sites in biomolecules. The term ‘biomolecular complexes of two or more biomolecules’ refers to complexes of two or more in some way associated biomolecules, which association between the molecules can be ‘fixed’ using a cross-linker according to the present invention. Thus, the mode of association of e.g. protein-protein complexes can be investigated. With the sequencing of the genomes, a large body of information is obtained on genes that encode proteins. In many cases, even a function of the protein encoded by the genes is known. However, nature is highly complex and many reaction in cells are accomplished by the interaction of proteins with other proteins, peptides, DNA or RNA. Actually, the fast majority of cellular protein is part of a protein complex containing more than one protein [A. C. Gavin et al., Nature 2002, 415:141; Y. Ho et al., Nature 2002, 415:180]. Non-limiting example are protein-protein complexes transiently formed during signal transduction, such as for example the MAP kinase signal transduction pathway. Moreover, complexes of proteins may be associated to RNA, such as spliceosomes, or to DNA such as the multienzyme complex involved in the nucleotide excision DNA repair pathway. A major question remaining to be answered is how such interactions are established, and how the interaction may affect processes within the cell. The present invention provides an easy tool to i) identify complexes that can be formed in a cell, ii) identify cross-linked sites in biomolecules, thereby charting interaction domains of the interacting partners in such complexes and iii) determine relative amounts of biomolecular complexes.

For these purposes it is important that the native structure of biomolecules during cross-linking is maintained, i.e. that harsh conditions causing denaturation of their structure are avoided during cross-linking. It is likely that this will only be achieved under conditions of limited cross-linking, in all likelihood resulting in a preparation containing a molecule population having a varying degree and site(s) of cross-links present.

In principle, the molecules to be cross-linked do not necessarily have to be biomolecules, but it is this application of the cross-linkers that provide the most significant advancement in the art.

Thus, in a preferred embodiment, the biomolecules are chosen from one or more of the group, consisting of protein, peptide, DNA, RNA, carbohydrates and lipids, or combinations thereof.

In yet a further aspect, the present invention provides for a method for preparing cross-linked fragments from cross-linked biomolecules, biomolecular complexes or mixtures thereof as defined above, said method comprising the step of fragmenting the cross-linked biomolecules, biomolecular complexes or mixtures thereof. Such fragments are advantageously prepared in order to obtain cross-linked species from the cross-linked biomolecules or biomolecular complexes of sufficient small size for further analysis.

Cross-linked fragments can be obtained by any method known in the art for cleaving large biomolecules into smaller fragments. Such fragmentation can e.g. be performed by chemical protein cleavage or treatment of proteins with proteolytic enzymes as to obtain smaller size fragments. Preferably, fragmentation is carried out by selective cleavage reagents, since resulting fragments can be more easily identified then fragments obtained by a-selective cleavage. Examples of specific chemical cleavage agents for proteins and peptides are CNBr, which cleaves proteins at methionine residues, and dilute acid at pH 2, which cleaves specifically at aspartate residues at high temperature, e.g. 108° C. Examples of specific proteases are trypsin, cleaving at lysine and arginine residues and Glu-C endoproteinase, cleaving at glutamate and, to a lesser extent, at aspartate residues. Efficient cleavage of cross-linked protein complexes can also be performed with pepsin, cleaving preferentially in hydrophobic segments with poor residue selectivity. In complex structures, identification of peptic peptides is more time-consuming then identification of peptides obtained by selective cleavage. Fragmentation can also be achieved by using a combination of two or more of such cleavage methods. For sequence specific cleavage of DNA in protein-DNA cross-links, restriction enzymes can be employed. However, for cross-linked protein-DNA or protein-RNA complexes, aspecific nucleases, such as DNase I and the RNase α-sarcin, can be employed to decrease the size of the cross-linked nucleic acid moiety to the part that is protected towards nuclease digestion by interaction with the protein. Similarly, for protein-polysaccharide complexes, several glycosylases can be employed to decrease the size of the polysaccharide moiety.

In yet a further aspect, the present invention relates to a method for cleaving of an azide-reducing agent-sensitive scissile amide bond in a portion of cross-linked biomolecules, biomolecular complexes or mixtures thereof as defined in any of claims 9 or 10, or cross-linked fragments as defined in claim 11, the carbonyl carbon atom of the amide bond being positioned 3 or 4 atoms from the azide group, and reducing of the azide group to an amide group in another portion of cross-linked biomolecules, biomolecular complexes or mixtures thereof as defined in any of claims 9 or 10, or cross-linked fragments as defined in claim 11, said method comprising the steps of:
A) providing cross-linked biomolecules, biomolecular complexes or mixtures thereof as defined in any of claims 9 or 10, or cross-linked fragments as defined in claim 11; and
B) subjecting the cross-linked biomolecules, biomolecular complexes or mixtures thereof, or cross-linked fragments of step A) to an azide-reducing agent in a protic solvent thereby cleaving the cross-link in a portion of the cross-linked biomolecules, biomolecular complexes or mixtures thereof, or cross-linked fragments, and reducing the azide group to an amine group in another portion of the cross-linked biomolecules, biomolecular complexes or mixtures thereof, or cross-linked fragments.

The present inventors anticipated that the two azide-reducing agent-induced reactions that were observed in azidohomoalanine-containing peptides (J. W. Back, O. David, G. Kramer, G. Masson, P. T. Kasper, L. J. de Koning, L. de Jong, J. H. van Maarseveen, C. G. de Koster, Angew. Chem. 2005, in press) would also occur in cross-linked fragments obtained after e.g. proteolytic digestion of protein complexes cross-linked with e.g. BACOX or NAG as indicated above. The first reaction is hydrolysis of one of the two amide bonds that had been formed between the protein and the cross-linker according to the present invention, e.g. BACOX or NAG, during cross-linking. The second of the two competing reactions induced by the azide-reducing agent is the conversion of the azide group in the cross-linker into an amine. These conversions can advantageously be used to determine the sites of cross-linking in complexes according to the present invention, as will be further explained below.

A “protic solvent” as herein used, refers to a solvent that is capable of donating a hydrogen atom for hydrogen bonding. This usually requires an NH or OH bond. Non-limiting examples thereof are aqueous solutions, such as water and several types of buffer solutions, and alcohols such as ethanol, nitriles such as acetonitrile, organic acids such as acetic acid, furanes such as tetrahydrofurane, formamides such as dimethylformamide and any mixture of these solvents. The protic solvent should allow for solubilisation of the biomolecules as well as the azide-reducing agent.

As herein used, the phrase “subjecting the cross-linked biomolecules, biomolecular complexes or mixtures thereof, or cross-linked fragments of step A) to an azide-reducing agent in a protic solvent” refers to a reaction that occurs between the cross-linked biomolecules, biomolecular complexes or mixtures thereof, or cross-linked fragments of step A) and an azide-reducing agent when these are simultaneously present in the protic solvent. The cross-linked biomolecules, biomolecular complexes or mixtures thereof, or cross-linked fragments of step A) will thus be subjected to the azide-reducing agent when a sample of cross-linked biomolecules, biomolecular complexes or mixtures thereof, or cross-linked fragments of step A) in protic solvent is mixed with the azide-reducing agent in the same protic solvent or a protic solvent that is miscible with the protic solvent used to solubilise the cross-linked biomolecules, biomolecular complexes or mixtures thereof, or cross-linked fragments of step A).

As herein used, the term azide-reducing agent refers to any compound that can ensure reduction of an azide, e.g. a H2/catalyst, tertiary phosphine, or a thiol-containing compound, preferably a tertiary phosphine or a thiol-containing compound.

The tertiary phosphine may be any phosphine, in particular those of formula III

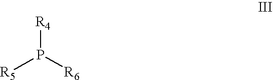

, wherein R₄, R₅ and R₆ are independently optionally substituted alkyl or aryl chains and may be the same or different. Of particular interest are a combination of R₄, R₅ and R₆ groups that render the phosphine soluble in a protic solvent, in particular in water. Non-limiting examples of R₄, R₅ and R₆ include carboxylic acids (e.g. propionic acid), alkylamines (e.g. propylamine) alkylhydroxyls (e.g. propanols), alkylsulfonyls, or alkylguanosyls. Such phosphines are well known in the art.

The thiol-containing compound may be any thiol-containing compound known in the art, such as 2-mercaptoethanol, dithiothreitol (DTT), etcetera. It was found that the reaction with dithiols such as DTT is more efficient than with monothiols such as 2-mercaptoethanol.

In a portion of the cross-linked biomolecules, biomolecular complexes or mixtures thereof, or cross-linked fragments of step A), the amide bond of which the carbonyl carbon atom is positioned 3 or 4 atoms from to the azide group is to be cleaved using the azide-reducing agent, thereby obtaining a free amine and a lactone. It should be noted that the lactone is easily hydrolysed at alkaline pH, or that lactones can be easily derivatized with suitable nucleophiles, e.g. amines or alcohols, (J. W. Back, O. David, G. Kramer, G. Masson, P. T. Kasper, L. J. de Koning, L. de Jong, J. H. van Maarseveen, C. G. de Koster, Angew. Chem. 2005, in press). In another portion of the cross-linked biomolecules, biomolecular complexes or mixtures thereof, or cross-linked fragments of step A) the azide group will be reduced to an amine group. The simultaneous occurrence of both reactions is important for the method for identifying cross-links, as will be disclosed further below.

In an embodiment of the method according to the present invention, the azide-reducing agent is chosen from the group, consisting of a tertiary phosphine and a thiol-containing compound. It was found that such azide-reducing agents perform particularly well in the method according to the present invention.

In a preferred embodiment, the tertiary phosphine is chosen from the group, consisting of tris(carboxyethyl)phosphine, tris(carboxypropyl)phosphine, tris(hydroxyethyl)phosphine, tris(hydroxypropyl)phosphine, tris(ethylamine)phosphine and tris(propylamine)phosphine. These phosphines are readily soluble in protic solvents at applicable pH values and are therefore the preferred phosphines to be used.

In another preferred embodiment, the tertiary phosphine is tris(carboxyethyl)phosphine (TCEP), as this compound is readily available at a relatively low cost price. It is expected that the trisalkylaminephosphines and the trisalkylhydroxylphosphines will be particularly effective at low pH.

In another embodiment of the above method, the thiol-containing compound is a dithiol-containing compound, as it was found that compounds comprising at least two thiol groups are more efficient in reducing the azide group than compounds comprising a single thiol group.

In case of using a thiol-containing compound, it is preferred that this compound is dithiotreitol, butanedithiol or propanedithiol, since these are easily available at a relatively low cost prize.

In a further preferred embodiment, the protic solvent is an aqueous solution. In such aqueous solution, protons are available for aiding the cleavage reaction. In the case of use of an aqueous solution as disclosed above, it is preferred that the azide-reducing agent is water-soluble such that reaction between the azide-reducing agent and the cross-linker according to the present invention is facilitated and most efficient.

In a preferred embodiment, step B) is carried out at a pH in the range of 3-10. The preferred pH is dependent on the protic solvent and azide-reducing agent used and the solubility of the phosphine used. One skilled in the art will readily be capable of determining a suitable system for carrying out the invention.

In a more preferred embodiment of the method according to the present invention, step B) is carried out at a pH in the range of 4-9, as in this pH range best results are obtained using an aqueous solution as a protic solvent and e.g. TCEP as a phosphine.

In yet a further aspect, the present invention relates to a method for identifying cross-links in one or more cross-linked biomolecules, biomolecular complexes or mixtures thereof obtainable by the method as disclosed above and/or cross-linked fragments obtainable by the method as defined above, said method comprising the step of using the cross-linked biomolecules, biomolecular complexes or mixtures thereof and/or cross-linked fragments thereof.

Identifying cross-links implies determination of the monomer sequences of the two biomolecules connected by a cross-link and assessment of the identity of the cross-linked monomer residues. Often this requires fragmentation of the cross-linked biomolecular complexes, determination of monomer sequences in the two cross-linked fragments and assessment of the cross-linked monomer residues in the fragments.

Said identifying can be conducted by any technique known in the art, but is preferably performed by mass spectrometric analysis using commercially available mass spectrometric equipment. One skilled in the art will be aware of suitable methods for performing such analyses. Such methods are e.g. described in E. de Hoffmann & V. Stroobant, Mass spectrometry; principles and applications. Wiley: Chichester, England 2002. An example of such suitable method and apparatus therefor is disclosed hereinafter.

In an embodiment, said method comprises the steps of:

a) providing said one or more cross-linked biomolecules, biomolecular complexes or mixtures thereof, and/or said cross-linked fragments;
b) optionally, fractionating said one or more cross-linked biomolecules, biomolecular complexes or mixtures thereof, and/or said cross-linked fragments into fractions comprising said one or more cross-linked biomolecules, biomolecular complexes or mixtures thereof, and/or said cross-linked fragments;
c) subjecting the cross-linked biomolecules, biomolecular complexes or mixtures thereof, and/or said cross-linked fragments of step a), or fractions thereof of step b) to a method as defined in claim 12 to obtain (a) reaction mixture(s);
d) identifying the cross-links by mass spectrometric analysis of the reaction mixture(s) of step c).

In step a), one or more cross-linked biomolecules, biomolecular complexes or mixtures thereof as defined above, and/or cross-linked fragments as defined above are provided. The cross-linked biomolecules, biomolecular complexes or mixtures thereof and/or the cross-linked fragments may be present in a mixture comprising other components, such as non-cross-linked biomolecules, biomolecular complexes and fragments and singly labelled biomolecules, i.e. biomolecules having one or more cross-linkers attached thereto but not being cross-linked to an identical or other biomolecule, and complexes and fragments thereof. This heterogeneity is the result of both the propensity of most functional end groups of cross-linkers to hydrolyse under conditions of cross-linking, which may lead to the formation of singly labelled biomolecules along with cross-linked biomolecules, and the selected conditions for partial cross-linking as argued above, implying that the cross-linking efficiency will not be 100%.

In step b) optionally said one or more cross-linked biomolecules, biomolecular complexes or mixtures thereof, and/or said cross-linked fragments are fractionated into fractions comprising said one or more cross-linked biomolecules, biomolecular complexes or mixtures thereof, and/or said cross-linked fragments. Said fractionation step serves to separate cross-linked species in the cross-linked biomolecules, biomolecular complexes or mixtures thereof and/or the cross-linked fragments from the corresponding unmodified or singly labelled species and to decrease the complexity of the sample. Separation of cross-linked species from corresponding singly labelled species is important for unambiguous identification of cross-links. This is related to the fact that azide-reducing cleavage products from cross-linked and singly labelled species are partially identical, complicating identification of cross-links. Moreover, depending on the exact position of the azide-reducing agent-sensitive scissile amide bond in cross-linked species, cleavage may result in formation of unmodified species, requiring separation of cross-linked species from pre-existing unmodified species.

Thus, the fractionation step is in particular performed in case of the provision in step a) of the cross-linked biomolecules, biomolecular complexes or mixtures thereof and the cross-linked fragments in a mixture with other components such as non-cross-linked biomolecules, biomolecular complexes and fragments and singly labelled biomolecules, and complexes and fragments thereof. In case substantially pure cross-linked biomolecules, biomolecular complexes or mixtures thereof, and/or said cross-linked fragments are provided in step a), such that substantially no unmodified or singly labelled species are present and the mixture of cross-linked biomolecules, biomolecular complexes and/or cross-linked fragments is not too complex, the fractionation step b) may be omitted.

Such fractionation may be achieved by any method known in the art, such as reversed phase chromatography, ion exchange chromatography, gel filtration, affinity chromatography or any other chromatographic fractionation technique, electrophoresis, and a combination of one or more of these fractionation techniques. The (final) fractionation is preferably carried out by reversed phase chromatography, since fractions obtained by this technique can generally be directly used for subsequent steps without requirement for further clean-up steps.

In case of fractionation, each of the subsequent steps may be conducted on each fraction.

In step c), the cross-linked biomolecules, biomolecular complexes or mixtures thereof, and/or said cross-linked fragments of step a), or fractions thereof of step b) are subjected to a method for cleavage and reduction of cross-linked biomolecules as disclosed above to obtain (a) reaction mixture(s). The further details of said cleavage and reduction are set forth above.

In step d), the cross-links are identified by mass spectrometric analysis of the reaction mixture(s) of step c). The mass spectrometric analysis of the reaction mixture(s) of step c) comprises several steps: 1) assigning the signals belonging to pairs of cleavage products of a cross-linked species, and 2) identifying the cross-linked species.

A crucial feature for the first step is that the sum of the masses of the cleavage products differs in a defined way, namely by 0.984 Da, from the mass of the intact cross-linked species in which the azide group is reduced to an amine group. Using mass spectrometry and application of this so-called connectivity rule allows assignment of the signals belonging to pairs of cleavage products that were cross-linked to each other, even in complicated mixtures. Recognition of relevant signals in the mass spectrum of step d) may be facilitated by comparing the mass spectrum of step d) with a mass spectrum obtained from the sample before addition of the azide-reducing agent, so obtained preceding step c). Such a mass spectrum lacks the signals of the cleavage products. The signals belonging to cross-linked or singly labelled species have shifted because of the reduction of the azide group to an amine group, causing a mass decrease of 25.99 Da.

In the second step standard proteomics techniques based on the known sequence of the genome using accurate mass measurement and/or tandem mass spectrometry either with the ‘bottom-up’ (peptide level) or ‘top-down’ (intact protein level) approach [M. Mann, R. C Hendrickson and A. Pandey, Annu. Rev. Biochem. 2001, 70, 437; B. Bogdanov and R. D. Smith, J. Mass Spectrom. 2005], are applied. This will reveal both the amino acid sequence of the cleavage products in case the biomolecules are proteins or peptides, thereby disclosing the identity of the cross-linked protein (in case of an intramolecular cross-link) or proteins (in case of an intermolecular cross-link), and, in most cases, will also reveal the cross-linked monomer residues.

For cross-linked samples of relative high complexity, such as peptide mixtures from large cross-linked biological assemblies or from mixtures of protein complexes it is preferable to separate the azide reducing agent-induced reaction products from the bulk of unmodified peptides in order to facilitate identification of cross-links. Cross-linked peptides, and thus azide-reducing agent-induced reaction products thereof, are usually of low abundance and can as such easily escape detection by mass spectrometric analysis in the context of a complicated peptide mixture composed predominantly of abundant unmodified peptides, in particular if the cross-linked species happen to have a relatively low ionization efficiency. Moreover, cross-linked peptides are on average larger than unmodified species, implying distribution of the mass signals over more isotopic peaks, thereby decreasing the signal to noise ratio. A powerful method to isolate azide-reducing agent-induced reaction products, including the intact, reduced, cross-link, is diagonal chromatography (K. Gevaert, J. Van Damme, M. Goethals, G. R. Thomas, B. Hoorelbeke, H. Demol, L. Martens, M. Puype, A. Staes, J. Vandekerckhove, Mol Cell Proteomics 2002, 1, 896). By diagonal chromatography, species with a specific reactivity that influences their chromatographic behaviour are sorted out of a mixture of unreactive compounds. In a diagonal chromatography experiment applied to a digest of cross-linked proteins, the peptide mixture is first separated in fractions by reversed phase chromatography. Individual fractions are then treated with the azide-reducing agent followed by applying the reaction mixtures separately on the same reversed phase column, to separate reacted species, i.e., cleavage and reduction products, from inert ones. Dependent on their hydrophobicity, cleavage products will in general have shorter or longer retention times than the parent compound. The amine of a cross-linked peptide is more polar than the parent azide, and therefore will have a shorter retention time. Mass analysis of shifted fractions, containing only cleavage and reduction products from singly labeled or cross-linked species, will enable identification of cross-links in a similar way as in step d). The name ‘diagonal chromatography’ refers to points on a diagonal line that will be obtained when the retention times of various unreacted (and unreactive) species in the first fractionation are plotted against those of the second fractionation. The points off this diagonal line belong to reacted species.

In such embodiment, the fractions of step a) or b) are subjected to fractionation, preferably by liquid chromatography. Obtained fractions are subjected to treatment with azide-reducing agent as in step c) and the reaction mixture fractions are subsequently separately fractionated using the same fractionation technique as used to obtain the fractions. Subsequently, only the shifted fractions of the last fractionation, i.e. only fractions containing reacted products are subjected to mass spectrometric analysis to identify cross-links.

In this embodiment said method comprises the steps of:
I) providing said one or more cross-linked biomolecules, biomolecular complexes or mixtures thereof, and/or said cross-linked fragments;
II) fractionating said one or more cross-linked biomolecules, biomolecular complexes or mixtures thereof, and/or said cross-linked fragments or said fractions into fractions;
III) subjecting the fractions of step II) to a method of cleavage and reduction as defined above to obtain reacted fractions;
IV) fractionating said reacted fractions using the same fractionation technique used in step III) to separate reacted products from non-reacted products to obtain one or more reacted product fractions;
V) identifying the cross-links by mass spectrometric analysis of the reacted product fractions of step IV).

In step I), one or more cross-linked biomolecules, biomolecular complexes or mixtures thereof as defined above, and/or cross-linked fragments as defined above are provided. The cross-linked biomolecules, biomolecular complexes or mixtures thereof and/or the cross-linked fragments may be present in a mixture comprising other components, such as non-cross-linked biomolecules, biomolecular complexes and fragments and singly labelled biomolecules, i.e. biomolecules having one or more cross-linkers attached thereto but not being cross-linked to another biomolecule, and complexes and fragments thereof. The consequences thereof are discussed above.

In step II) said one or more cross-linked biomolecules, biomolecular complexes or mixtures thereof, and/or said cross-linked fragments are fractionated into fractions. Said fractionation step serves both to reduce the complexity of the sample and to separate cross-linked species in the cross-linked biomolecules, biomolecular complexes or mixtures thereof and/or the cross-linked fragments from the corresponding unmodified or singly labelled species.

Thus, the fractionation step is in particular performed in case the sample obtained in step I) is too complex to allow adequate cross-link analysis according to subsequent steps.

Such fractionation may be achieved by any method known in the art, such as reversed phase chromatography, ion exchange chromatography, gel filtration, affinity chromatography or any other chromatographic fractionation technique, and electrophoresis, or a combination of one or more of these fractionation techniques. The fractionation is preferably carried out by reversed phase chromatography, for reasons indicated below in respect of step IV).

In case of highly complex samples, is preferred to use a pre-fractionation step preceding step II) in order to perform a first complexity reduction. When reversed chromatography is used for the diagonal separation in the step II), the preferred pre-fractionation technique is, or includes, ion exchange chromatography, since it has been shown that the combination of ion exchange chromatography and reversed phase chromatography enables adequate fractionation of complex peptide mixtures (e.g., C. Delahunty & J. R. Yates, Methods 2005, 35, 248.

In step III), the fractions of step II) are subjected to a method of cleavage and reduction as defined above to obtain reacted fractions. The further details of the cleavage method are set forth above.

In step IV), said reacted fractions of step III) are fractionated using the same fractionation technique used in step II) to separate reacted products from non-reacted products to obtain one or more reacted product fractions. This fractionation step may be carried out as is discussed above, and is preferably carried out using reversed phase chromatography, since reacted products can be adequately separated from non-reacted-products by this technique. Moreover, fractions obtained by this technique do not require further clean-up steps for analysis by mass spectrometry.

In step V) the cross-links are identified by mass spectrometric analysis of the reacted product fractions of step IV). To this end the reacted product fractions are subjected to mass spectrometric analysis to identify cross-links in a similar fashion as disclosed above.

For reasons discussed above, the biomolecules are preferably chosen from one or more of the group, consisting of protein, peptide, DNA, RNA, carbohydrates, and lipids or combinations thereof. The biomolecules are most preferably proteins.

Preferably, step b) step II and/or step IV) are carried out by a chromatographic or electrophoretic fractionation technique or by one or more combinations of these techniques. These techniques are most suited to provide suitable fractionation.

In a final aspect the present invention provides for a method for determining relative amounts of cross-links in a biomolecule or biomolecular complex in two or more samples, said method comprising the step of using at least a first cross-linker and a second cross-linker as defined in any of claims 1-7, said first and second cross-linker being identical in chemical composition and structure, and said first cross-linker or second cross-linker being enriched in one or more isotopes resulting in a molar mass difference between said first and second cross-linker.

According to the present invention, one of the two samples is cross-linked with a ‘light’ version of the cross-linker, for example the first cross-linker, while the other preparation is cross-linked with a ‘heavy version’ of the same cross-linker, e.g. the second cross-linker, in which for example one or more the elements ¹H, ¹²C, ¹⁴N or ¹⁶O have been replaced by ²H, ¹³C, ¹⁵N or ¹⁸O, respectively, to give a molar mass difference.

Preferably, the isotopes to be used in these experiments are ¹³C, ¹⁵N or ¹⁸O, as the use of these isotopes give no difference in retention time with reversed phase liquid chromatography that may be used to fractionate the samples.

The way of determining relative quantities of particular cross-links is as follows: two or more samples to be compared are cross-linked with a light and heavy version of the cross-linker, respectively (first and second cross-linker). Then equivalent amounts of the cross-linked samples are mixed and subjected to the analysis method for identifying cross-links as described above. After identification, the relative amount of each cross-link is determined from the ratio of the areas of peaks in mass spectra corresponding to the “heavy” and the “light” cross-linked biomolecules or fragments thereof or, after treatment with azide-reducing agents, to the reduced form of the cross-linked biomolecules or fragments thereof, respectively.

Preferably, the molar mass difference between said first and second cross-linker is at least 2 Da, more preferably at least 4, yet more preferably at least 6 Da, most preferably at least 8 Da, as such molar mass difference gives sufficient peak resolution in most mass spectrometers to enable determination of accurate peak area ratios of signals from light and heavy isotopically labelled compounds. One skilled in the art will readily be able to determine the molar mass difference required to obtain sufficient peak resolution in a specific mass spectrometer.

It is trivial whether the first or second cross-linker has a higher molar mass, as long as they display a molar mass difference, such that it can be inferred from which sample the biomolecule or biomolecular complex is derived.

The two or more samples may be any samples, but are preferably biological samples. The method is advantageously designed to compare the formation of biomolecular complexes in cells, e.g. as a function of time during the cell cycle or as a result of a specific stimulus. A useful application of such analysis would e.g. be an investigation of the change in pattern of protein-protein interactions in a cell in time following ligand binding to a receptor and the concomitant activation of a connected signal transduction pathway. One could prepare two cell extracts: for example, an extract at t=0 and an extract on t=x. One of the extracts could be cross-linked with a ‘light’ version of the cross-linker according to the present invention, whereas the other extract could be cross-linked with a ‘heavy’ version of the cross-linker according to the present invention. Equivalent amounts of the cross-linked extracts can then be mixed and processed as described below. Each cross-link formed in the extracts at either t=0 or t=x provides two signals in a mass spectrum with a molar mass difference corresponding with the molar mass difference between the ‘light’ and ‘heavy’ version of the cross-linker. The intensity of these signals provide a measure for the amount present, and these can be compared to deduce increases or decreases in biomolecular complexes in the extracts.

In an embodiment, said method comprises the steps of:
1) providing a first and second sample comprising one or more biomolecules, biomolecular complexes, or mixtures thereof;
2) preparing a first and a second cross-linked sample comprising one or more cross-linked biomolecules, biomolecular complexes, or mixtures thereof by cross-linking of said first sample with said first cross-linker, and of said second sample with said second cross-linker using the method as defined above and in any of claims 9 or 10;
3) combining said first and second cross-linked sample to obtain a combined sample;
4) optionally, fragmenting said combined sample using the method as defined in claim 11 to obtain a fragmented combined sample;
5) performing steps b)-d) as defined above or steps II)-V) as defined above on the combined sample of step 3) or the fragmented combined sample of step 4);
6) determining the relative amount of each cross-link from the ratio of areas of the relevant peaks in mass spectra.

In step 1) a first and second sample comprising one or more biomolecules, biomolecular complexes, or mixtures thereof are provided.

In step 2) a first and a second cross-linked sample comprising one or more cross-linked biomolecules, biomolecular complexes, or mixtures thereof are prepared by cross-linking of said first sample with said first cross-linker, and of said second sample with said second cross-linker using the method as defined above and in any of claims 9 or 10.

In step 3) said first and second cross-linked sample are combined to obtain a combined sample. Cross-links from the first and second cross-linked samples can be distinguished by the molar mass difference between the first and second cross-linker used to prepare the cross-linked samples. Preferably, the first and second cross-linked samples are combined in a known ratio such that the amounts can ultimately be correlated. It is preferred that equivalent amounts of samples are combined such that the relative amounts of cross-links in a first and second sample can be directly correlated. Equivalent amounts are e.g. achieved by selecting equal amounts of cells for preparing cell-extracts.

In step 4), said combined sample is optionally fragmented using the method as defined above and in claim 11 to obtain a fragmented combined sample. Such fragmentation can be performed in order to obtain fragments of a suitable size as is discussed above.

In step 5) steps b)-d) as defined above or steps II)-V) as defined above are performed on the combined sample of step 3) or the fragmented combined sample of step 4). In case of cell extracts, it is preferred that steps II)-V) as defined above are conducted, since mass spectrometric identification and quantification of cross-links in such complex samples requires adequate fractionation in order to reduce the complexity of the samples to be analysed.

In step 6) the relative amount of each cross-link is determined from the ratio of the areas of the relevant peaks in mass spectra.

The cleavage and reduction reactions of cross-linked and singly labelled peptides and the separation of reaction products from parent compounds by reversed phase chromatography is disclosed in detail below with reference to the cross-linkers BACOX and NAG, the azide-reducing agent TCEP, and the peptide neurotensin. Experimental details are set forth in the examples section.

As discussed above, it was anticipated that the two azide-reducing reactions that were observed in azidohomoalanine-containing peptides (J. W. Back, O. David, G. Kramer, G. Masson, P. T. Kasper, L. J. de Koning, L. de Jong, J. H. van Maarseveen, C. G. de Koster, Angew. Chem. 2005, in press) would also occur in cross-linked peptides obtained after e.g. proteolytic digestion of protein complexes cross-linked with e.g. BACOX or NAG. The first reaction is hydrolysis of one of the two amide bonds that had been formed between the protein and the cross-linker according to the present invention, e.g. BACOX or NAG, during cross-linking (FIGS. 1B and 1C, 3a, 3b, marked amides). Upon cleavage of this amide bond in a cross-linked peptide, one of the two peptides in the cross-link is released unmodified, while the other is left modified with the cross-linker of which the free end is converted into a lactone. This modification adds 197.069 Da in the case of BACOX and 112.016 Da in the case of NAG to the mass of the peptide. Since the cross-linker can react with the protein in two orientations, the TCEP-induced cleavage reaction yields the two peptides of the cross-link both in their unmodified (see FIG. 1B; 4a, 5a, (BACOX (1)) and 4b and 5b (see FIG. 1C; NAG (2))) and modified forms (see FIG. 1B; 6a, 7a (BACOX (1)) and 6b, 7b (see FIG. 1C; NAG (2)). The second of the two competing reactions induced by TCEP is the conversion of the azide group in the cross-linker into an amine (see FIGS. 1B and 1C; 8a, 8b). This modification adds 196.085 Da in case of BACOX and 111.032 Da in case of NAG to the sum of the masses of the two connecting peptides. So, the mass of this modified intact cross-linked peptide is 0.984 Da (i.e., 197.069-196. 085 or 112.016-111.032) less than the sum of the masses of the two cleavage products of each isomer. This is the connectivity rule of cross-linked peptides. A major feature of BACOX and NAG is that application of the connectivity rule to a mixture of e.g. TCEP-treated cross-linked peptides enables unambiguous identification of the two TCEP-induced cleavage products derived from one isomer of a particular cross-link.

Peptides with internal cross-links (see FIG. 1D; 9) yield only two pairs of isomers (see FIG. 1D; 10, 11) in the presence of TCEP, with a mutual mass difference of 0.984 Da, (as shown in FIG. 1 for BACOX (1)). One of the two isomers (see FIG. 1E, 12) of singly modified peptides yields the unmodified peptide (see FIG. 1E, 13) as a result of cleavage of the amide bond by which the cross-linker is connected to the peptide, and the amine (see FIG. 1E, 15). The other isomer gives rise to the amine as well and the lactone form of the cross-linker-conjugated peptide (see FIG. 1E, 14). The mass differences with the unmodified peptide of the amine and the lactone are 214.095 and 197.069 Da, respectively, for BACOX and 129.046 and 112.016 Da, respectively, for NAG.

Thanks to these characteristic mass differences, cross-linked and singly labeled peptides can be recognized by comparing the mass, e.g. mass spectra, before and after reaction with TCEP of fractionated peptides, e.g. by means of reversed phase, to separate cross-linked peptides from their singly labeled counter parts. The ability of the cross-linkers according to the present invention, e.g. BACOX and NAG, to easily distinguish between cross-links and singly labeled species is of great practical use. Another major feature of the reactions induced by the azide-reducing agent, e.g. TCEP, is the appearance of unmodified peptides from cross-linked species. These peptides can be easily identified, e.g. by tandem mass spectrometry (MS/MS) or by accurate mass measurement alone. Furthermore, MS/MS of the lactone will identify the amino acid involved in the cross-link and MS/MS of the intact (unmodified or reduced) cross-link will validate the identification obtained by application of the connectivity rules.

By these features alone the cross-linkers according to the present invention, exemplified by BACOX and NAG, are far superior to other cross-linkers designed in the art thus far. The added value of the azide group-containing cross-linkers becomes increasingly apparent when mass analysis of TCEP-induced cleavage and reduction products is combined with fractionation, e.g. diagonal chromatography, to sort out cross-linked and singly labeled peptides from the bulk of unmodified peptides.

In a diagonal chromatography experiment (W. H. Cruickshank, B. L. Malchy & H. Kaplan, Can. J. Biochem., 1974, 52, 1013; K. Gevaert, J. van Damme, M. Goethals, G. R. Thomas, B. Hoorelbeke, H. Demol, L. Martens, M. Puype, A. Staes & J. Vandekerckhove, Mol. Cell. Proteomic. 2002, 1, 896; P. R. Liu, C L Feasly and F E Regnier, J. Chromatogr. A. 2004, 1047, 221-227), peptide mixtures from e.g. protease-digested protein complexes cross-linked with cross-linkers such as BACOX or NAG are first fractionated, e.g. by reversed phase chromatography (RPC). Subsequently, each fraction is treated with an azide-reducing agent, e.g. exemplified by TCEP, thereby modifying only peptides modified by the azide-reducing agent, e.g. BACOX or NAG, followed by separate subjection to the same reversed phase chromatography conditions. Cleavage products are expected to have in general different retention times with RPC as compared with the cross-linked species from which they are formed. The reduced form of the cross-linked peptide is more polar than the parent compound and will therefore elute earlier. So, the TCEP reaction products will separate from the bulk of unmodified peptides by this second chromatographic fractionation. The purification of cross-linked and singly labeled species and their TCEP-induced cleavage products from the bulk of unlabeled material greatly facilitates recognition and identification of constituting peptides by mass analysis and application of the connectivity rules. This powerful method will allow unambiguous identification of cross-linked sites in large protein complexes, even in complicated mixtures.

The present inventors investigated whether the two TCEP-induced reactions that were recently observed in azidohomoalanine-containing peptides (J. W. Back, O. David, G. Kramer, G. Masson, P. T. Kasper, L. J. de Koning, L. de Jong, J. H. van Maarseveen & C. G. de Koster, Angew. Chem. 2005, in press) also occur in peptides cross-linked or singly labeled with an azide group-containing cross-linker according to the present invention, e.g. BACOX or NAG. For this purpose neurotensin was subjected to cross-linking as described in example 3. Neurotensin (ZLYENKPRRPYIL, in which Z is pyroglutamate) possesses one free amine able to react with e.g. BACOX or NAG. After a primary reaction, the other reactive half of the cross-linker can react with a second neurotensin molecule. Also singly labeled species were present in the reaction mixtures. Purified cross-linked species were subjected to reaction with TCEP. Both the expected cleavage products and the reduced forms of the cross-linked species were formed in the presence of TCEP as judged from mass spectra of TCEP-induced reaction products (FIG. 3 and table 1) Moreover, no other signals were present in these mass spectra, pointing to the selectivity of the azide group and the specificity of the reactions. Mass spectra of TCEP-induced reaction products of the neurotensin species singly labeled with BACOX or NAG contained signals with m/z values corresponding to the free neurotensin, the amines of cross-linker-modified neurotensin and their corresponding lactones. The fact that both free neurotensin and the lactones were formed indicated that the two theoretical isomers of singly labeled neurotensin had been formed with both BACOX and NAG.

Mapping of cross-links introduced by BACOX and NAG in complex structures would be greatly facilitated if one would be able to separate TCEP-induced reaction products of cross-linked peptides from the bulk of unmodified peptides. To sound out whether diagonal chromatography could be used to separate cross-linker-labeled peptides from unlabelled species, the present inventors investigated to which extent the retention times in reversed phase HPLC of TCEP-induced reaction products of neurotensin cross-linked with BACOX differed from those of the parent compounds. It appeared that the free peptide, the lactone, and the reduced form of the intact cross-link eluted 7.84, 4.75 and 2.40 minutes earlier, respectively, than the unmodified cross-linked peptide (results not shown). The chromatographic resolution under these conditions is 0.5 min (full width at half peak maximum; results not shown). A retention time difference of three times the resolution, i.e., 1.5 min, is considered to be sufficient for effective separation of modified products from unmodified substances by diagonal chromatography. Even the smallest difference in retention time, i.e., 2.40 min, was much larger than this 1.5 min. This implies that diagonal chromatography is potentially a powerful way to sort TCEP-induced reaction products of peptides cross-linked or otherwise labelled by BACOX and NAG out of a peptide mixture obtained by proteolytic digestion of a cross-linked protein complex.

In conclusion, the present inventors describe here the properties of a new type of simple cross-linker provided with an azido group that is aptly positioned relative to an amide bond that is formed during cross-linking between amine groups or is already present in the cross-linker as such. The present inventors demonstrate that the products formed by treatment of cross-linked peptides with an azide-reducing agent such as a phosphine or thiol can provide an easy clue towards the peptide identity of the cross-link. This feature enables easy assessment of cross-linked sites in protein complexes, e.g. by MS/MS. However, the real bonus of these cross-linkers is in the combination of mass analysis of azide-reducing agent-induced reaction products with diagonal chromatography to sort out cross-linked and singly labelled peptides from the bulk of unmodified peptides. This combination will allow rapid and unambiguous identification of cross-linked sites in large protein complexes, even in complicated mixtures.

The present invention will be further illustrated by way of figures and examples, which are in no way to be construed as to limit the scope of the appended claims, wherein

FIG. 1A shows structures of the cross-linkers BACOX (1): bis(sulfosuccinimidyl) 5-(3-azido-1-carboxypropylamino)-5-oxopentanoate (BACOX); and NAG (2): bis(succinimidyl) 2-azido-glutarate (NAG);

FIG. 1B shows cross-linked and singly labelled peptides using BACOX as a cross-linker, wherein 3a denotes the two isomers of cross-linked peptides using BACOX as a cross-linker, and 4a-8a denote the six TCEP-induced reaction products that are formed from 3a. 4a denotes free peptide 1; 5a, free peptide 2; 6a, peptide 1 modified by the lactone derivative of 1; 7a, peptide 2 modified by the lactone derivative of 1; and 8a, the two isomers of the reduced form of 3a;

FIG. 1C shows cross-linked and singly labeled peptides using NAG as a cross-linker, wherein 3b denotes the two isomers of a cross-linked peptide using NAG as a cross-linker; and 4b-8b denote the six TCEP-induced cleavage products that are formed from 3b. 4b denotes free peptide 1; 5b, free peptide 2; 6b, peptide 1 modified by the lactone derivative of 2; 7b, peptide 2 modified by the lactone derivative of 2; and 8b, the two isomers of the reduced form of 3b.

FIG. 1D shows the TCEP-induced cleavage products, wherein 9 denotes an internally cross-linked peptide using BACOX as the cross-linker; 10 denotes the TCEP-induced cleavage product of 9; and 11 denotes the TCEP-induced reduction product of 9.

FIG. 1E denotes: 12, the two isomers of a singly modified peptide using BACOX as cross-linker. After or before a primary reaction of the cross-linker with an amine, the other activated ester bond has been hydrolyzed, resulting in formation of a terminal carboxylic acid group; 13, TCEP-induced cleavage product of the lower isomer of 12; 14, TCEP-induced cleavage product of the upper isomer of 12; and 15, the two isomers of the TCEP-induced reduction products of 12.

FIG. 2 shows the mechanism proposed for the reaction: Phosphines add to the electron deficient centre of the azide 1 initially forming intermediate 2, that may either hydrolyze through postulated intermediate 3 into the triazene 4 or fragment into aza-ylide 5. Triazenes have previously been shown react through an S_{N}2 reaction with suitable nucleophiles of either intra- or intermolecular origin. It is worthy to mention that at elevated pH conversion of 1 to homoserine without peptide cleavage occurs, indicative of S_{N}2 attack by OH⁻. The protonated triazene 6 enables an energetically favorable five-membered membered ring closure resulting in cyclic imido ester 7, a pathway analogous to the cyanogen bromide induced cleavage of the methionine peptide bond. Finally, hydrolysis of the imido ester 7 produces the homoserine lactone 8 and amine 9. This mechanism is supported by the ¹⁸O labeling experimental results described below which allow the carbonyl oxygen to be conserved in the lactone.

Alternatively, the phosphine activated azide, aza-ylide 5 can be reduced to amine or may become protonated generating intermediate 11 which via intramolecular S_{N}2 displacement involving the amide oxygen atom yields the common intermediate imido-ester 7, that will then again hydrolyze into 8 and 9.

The cleavage induced by (di-)sulfides is presumed to be initiated by attack of the thiolate anion at either the α- or—as depicted—the γ azide nitrogen (intermediate 10), that after elimination of a cyclic disulfide gives the triazene 4, which follows the pathways to 7, or can be reduced to DAB.

FIG. 3 shows MALDI-TOF mass spectra of TCEP-induced reaction products of cross-linked neurotensin. Neurotensin was cross-linked with 1 (A) or 2 (B) as described below in example 3. Cross-linked species were purified by reversed phase HPLC, treated with TCEP and analyzed by mass spectrometry as described below in examples 3 and 4. Signals corresponding to the following protonated species were observed (see Table 1); free neurotensin (e), neurotensin modified by the cross-linker in the form of a lactone (g, i), and cross-linked reduced neurotensin (f, h). Both protonated and sodiated ions are present differing 22 m/z. The reduced forms of neurotensin cross-linked both with NAG and BACOX are also present as a doubly charged protonated species. The neurotensin preparation contained a minor compound lacking one proline residue. This minor species could not be separated by reversed phase HPLC from full length neurotensin. The minor peaks (arrows) at m/z 97 from the main peaks can be attributed to the presence of this shorter form of neurotensin. The signals from the lactone of this form in panel A are overlapping with those from the doubly charged reduced form.

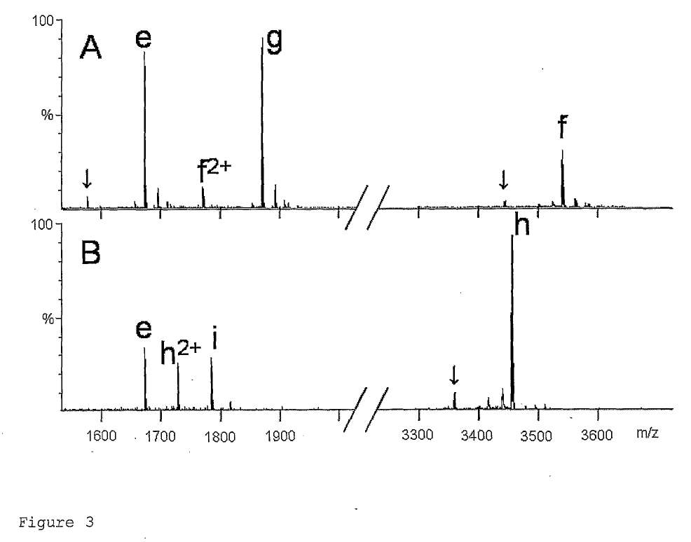

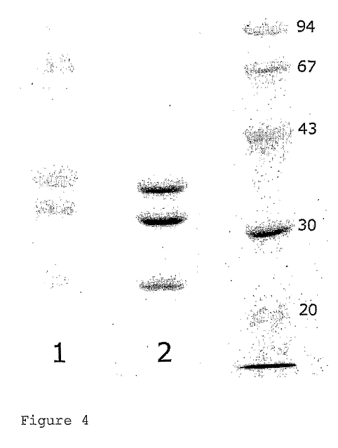

FIG. 4 demonstrates effective crosslinking of the protein complex consisting of the NK1 domain of HGF/SF (˜21.7 kD), the α-chain of Met receptor (˜33.5 kD) and the sema-domain of Met receptor β-chain (˜31 kD). Shown is a Coomassie Brilliant Blue stained polyacrylamide gel run in the presence of sodium dodecylsulphate and 2-mercaptoethanol. Lane 1 shows the complex modified with 10 mM BACOX, lane 2 shows negative control (sample treated in absence of BACOX). The lane at the right contains marker proteins of which the size in kD is indicated. The high molecular weight bands in lane 1 are the result of cross-linking between the different subunits in the complex.

### EXAMPLES

### Example 1

### Synthesis of BACOX

4.183 g (19.2 mmol) Boc-L-diaminobutyric acid (Chem-Impex Int., IL, USA) was converted to Boc-azidohomolanine as described in the procedure for the diazo-transfer reaction (O. David, W. J. Meester, H. Bieraugel, H. E. Schoemaker, H. Hiemstra, J. H. van Maarseveen, Angew. Chem. Int. Ed. Engl., 2003, 42, 4373).

Boc-azidohomoalanine was dissolved in dry DMF (17 ml) and N,N carbodiimidazole (3.11 g (19.2 mmol)) was added. The mixture was stirred for one hour at 70° C. After cooling down to room temperature 1.56 ml (38.4 mmol) of methanol and 2.87 ml (19.2 mmol) of DBU (1,8-diazabicyclo-(5.4.0) undec-7-ene) were added. The reaction solution was stirred at 70° C. for 60 h. DMF was removed by running three times ethyl acetate-water extraction. The organic layer was dried over Na₂SO₄ and evaporated. The amino group was deprotected (Boc group removed) in 65 ml 4M HCl in dioxane at room temperature. The completion of the reaction was monitored by TLC. HCl and dioxane were removed in rotary evaporator.

Met-azidohomoalanine and glutaric anhydride were dissolved in dioxane and both solutions were mixed. Two equivalents of NaHCO₃ were added. Dioxane was removed in a rotavap and the product was purified by chromatography on silica gel column. 5-(4-azido-1-methoxy-1-oxobutan-2-ylamino)-5-oxopentanoic acid (172 mg, 0.63 mmol) was dissolved in 1 ml methanol and mixed with 3.6 ml 1M NaOH. The reaction was monitored by TLC. Methanol was removed in a rotary evaporator and the product (5-(3-azido-1-carboxypropylamino)-5-oxopentanoic acid) was extracted with ethyl acetate from the reaction mixture acidified with HCl to pH 1. To a closed reaction vessel containing solution of 156 mg (0.63 mmol) 5-(3-azido-1-carboxypropylamino)-5-oxopentanoic acid and 342 mg (1.57 mmol) N-hydroxysulfosuccinimide in 5 ml dry dimethylformamide (DMF) solution of 346.5 mg (1.68 mmol) N,N′-dicyclohexylcarbodiimide in 1.5 ml dry DMF was added. The reaction solution was stirred at room temperature for three days. Precipitated dicyclohexylurea was filtered off and the supernatant was added to 100 ml ethyl acetate. The precipitated product was separated on the glass filter, washed with ethyl acetate and dried under vacuum.

### Example 2

### Synthesis of bis(succinimidyl) 2-azido-glutarate (NAG)

NAG was synthesized by coupling 2-azidopentanedioic acid with N-hydroxysuccinimid in the presence of dicyclohexylcarbodiimide. Azidopentanoic acid was synthesized from L-glutamic acid and tryflyl azide. Ten equivalents of tryflyl azide (J. T. Lundquist, J. C. Pelletier, Org. Lett. 2001, 3, 781) in CH₂Cl₂ were added to a stirred solution of L-glutamic acid (1.006 g; 6.836 mmol), K₂CO₃ (2.824 g; 20.4356 mmol) and Cu^{II}SO₄.5H₂O (78.5 μmol; 19.6 mg) in MeOH (45 ml) and H₂O (22 ml) and stirred overnight. Subsequently, the organic solvents were removed and the residual slurry was diluted with H₂O (100 ml). This was acidified to pH 6 with a 5% HCl solution, diluted with 0.25 M, pH 6.2 phosphate-buffer (125 ml) and extracted with EtOAc (4×60 ml) to remove the sulfonamide by-product. The aqueous phase then acidified to pH 2 using concentrated HCl. The product was obtained from EtOAc extractions (3×60 ml). The organic extracts were combined, dried (MgSO₄) and evaporated to dryness to give 1.037 g of the light yellow oil in 88% yield, with no need for further purification. ¹H-NMR (400 MHz, CDCl₃): δ 4.10 (t, 1H, J=6.3 Hz), 2.6 (m, 4H), 2.22 (q, 2H, J=6.4 Hz); IR (neat): 3445, 2926, 2113, 1740, 1711.

2-Azido-pentanedioic acid (1.037 g; 5.991 mmol) and DCC (2.738 g; 13.27 mmol) were dissolved in distilled CH₂Cl₂ (130 ml) under a N₂ atmosphere, and after addition of N-Hydroxysuccinimide (1.397 g; 12.05 mmol) and a catalytic amount of DMAP, this was stirred overnight. Et₂O was added to the reaction mixture to precipitate the DCU, which was removed by filtration, and the solvents were removed under reduced pressure, after which the residue was dissolved in EtOAc and a few ml of Et₂O. This was filtered and the filtrate was evaporated to dryness to give 2.188 g of the light brown oil in >95% yield. ¹H-NMR (400 MHz, CDCl₃): δ 4.10 (dd, 1H, J=6.3, 6.8 Hz), 2.9 (m, 2H), 2.81 (s, 8H), 2.25-2.45 (m, 4H); IR (neat): 2946, 2114, 1819, 1784, 1742, 1708.

### Example 3

### Synthesis of Cross-Linked Neurotensin

Neurotensin was purchased from Sigma. 10.15 μl of a 1 mM stock solution of neurotensin in water was placed in a vial and dried in vacuum centrifuge. To the dried peptide 25 μl 0.24 μM BACOX or NAG in dry DMF was added. The reaction mixture was incubated overnight in a closed vial at room temperature. DMF was removed in a vacuum centrifuge prior to chromatographic separation. Under these conditions the major compound in de reaction mixture is cross-linked neurotensin. Also singly labeled neurotensin has been formed under these conditions. Cross-linked neurotensin was purified by reversed phase HPLC.

### Reversed Phase HPLC

For reversed phase HPLC a Jupiter Proteo C12 column (inner diameter 2 mm, length 150 mm, Phenomenex, Torrance, USA) was used operated on a SMART system provided with a fraction collector (AmershamPharmacia, Uppsala, Sweden). A constant flow rate of 80 μl/min was maintained. Following injection of the sample, the column was rinsed with 0.1% trifluoroacetic acid in water (solvent A) for 10 min followed by a linear gradient to 50% acetonitrile in 0.1% trifluoroacetic acid (solvent B) over 75 min. Fractions of 1 min were collected starting at 10% acetonitrile and analyzed by mass spectrometry to identify peaks containing singly labelled neurotensin and cross-linked neurotensin.

### Example 4

### Reaction with TCEP

Samples of cross-linked and singly modified neurotensin were treated overnight at room temperature with 100 mM TCEP in 0.5 M sodium acetate pH 4.5. Samples were desalted on ZipTip C₁₈ (Millipore, Bedford, USA).

### Mass Spectrometry

Reflectron MALDI-TOF mass spectra were recorded on a Micromass TofSpec 2EC (Micromass, Whyttenshawe, UK).

### Example 5

### Cross-Linking Protein Complexes with BACOX

BACOX was dissolved in 20 mM NaPᵢ (pH 6.5) immediately before addition to the 0.5 g/l protein complex solution in 150 mM NaCl and 50 mM NaPᵢ (pH 7.4). The reaction mixture was incubated at room temperature for 1 h in the presence of 10 mM BACOX. The extent of cross-linking was assessed by the appearance of new high molecular weight bands in cross-linked complexes subjected to polyacrylamide gel electrophoresis in the presence of sodium dodecyl sulphate under non-reducing conditions (U. K. Laemmli, Nature 1970, 227, 680).

TABLE 1
Overview of theoretical and observed masses of cross-linked
products of neurotensin (NT) with BACOX and NAG and their TCEP-
induced reaction products. For experimental details see
examples 3 and 4. Code, small letters refer to peaks in mass
spectra shown in FIG. 3

| | | | | | TCEP-induced |
|---|---|---|---|---|---|
| | | | | | reaction |
| | | | | theoretical | product from |
| peptide | Code | experimental m/z | Δ ppm | m/z | (code) |
| NT | e | 1672.782 | −81 | 1672.91695 | A, B, C, D |
| NT-BACOX-NT | A | 3566.448 | −127 | 3566.90192 | — |
| NT-BACOX (red)-NT | f | 3540.426 | −137 | 3540.91142 | A |
| NT- | g | 1869.826 | −85 | 1869.98575 | A, B |
| BACOX (lactone) | | | | | |
| NT-BACOX | B | 1912.734 | −141 | 1913.00280 | — |
| NT-BACOX (red) | — | 1887.023 | 6 | 1887.01230 | B |
| NT-NAG-NT | C | 3481.777 | −21 | 3481.84915 | — |
| NT-NAG (red)-NT | h | 3455.954 | 28 | 3455.85866 | C |
| NT-NAG (lactone) | i | 1784.883 | −28 | 1784.93300 | C, D |
| NT-NAG | D | 1827.869 | −44 | 1827.95004 | — |
| NT-NAG (red) | — | 1801.938 | −12 | 1801.95955 | D |

1. A cross-linker having the following structure: 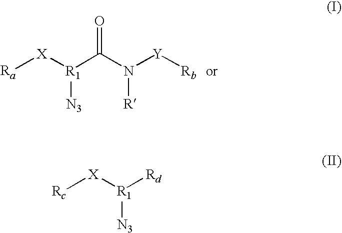
, wherein
Rₐ, R_{b}, and R_{c} are functional end groups reactive with a functional cross-linking group on a biomolecule and Rₐ and R_{b} can be the same or different;
R_{d} is a functional end group reactive with an N-containing functional cross-linking group on a biomolecule as to form an amide bond;
X and Y are optional and can be any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether, polyacetal, polycarbonate, polysaccharide, polyamide, polypeptide, polyurethane and polyester moieties, and can be the same or different;
R₁ can be any (optionally substituted) moiety having 3 or 4 atoms as to provide a distance of 3 or 4 atoms between the azide group —N₃ and the carbonyl carbon atom of the amide bond; and
R′ can be hydrogen, or can be any, optionally substituted, branched or unbranched alkyl, aryl, heteroalkyl, heteroaryl, alkenyl, alkynyl, cycloalkyl, arylalkyl, heteroarylalkyl, alkoxy, cycloalkylmethoxy and cycloalkylalkoxy, polyether, polyacetal, polycarbonate, polysaccharide, polyamide, polypeptide, polyurethane and polyester.

2. The cross-linker according to claim 1, wherein Rₐ, R_{b}, and R_{c}, can be the same or different and are chosen from the group, consisting of α-haloacetyl compounds, N-maleimide derivatives, mercurials, aryl halides, aldehydes, ketones, isocyanates, isothiocyanates, imidoesters, acid halides, acid anhydride, N-hydroxysuccinimidyl and other activated esters, N-acetylimidazole, diazoacetate esters, diazoacetamides, carbodiimides, diazonium compounds, dicarbonyl reagents, epoxides, and aryl azides.

3. The cross-linker according to claim 1, wherein R′ is hydrogen.

4. The cross-linker of claim 1, wherein the structure comprises bis(sulfosuccinimidyl) 5-(3-azido-1-carboxypropylamino)-5-oxopentanoate.

5. The cross-linker of claim 1, wherein the structure comprises bis(succinimidyl) 2-azido-glutarate.

6. The cross-linker according to claim 1, further comprising one or more isotopes of an element.

7. The cross-linker according to claim 6, wherein the one or more isotopes of an element are chosen from the group consisting of ²H, ¹³C, ¹⁵N and/or ¹⁸O.

8. A method for preparing a cross-linker as defined in claim 1, said method comprising the use of an azide-functionalized spacer.

9. A method for preparing one or more cross-linked biomolecules, biomolecular complexes of two or more biomolecules or mixtures thereof, said method comprising the step of using the cross-linker as defined in claim 1.

10. The method according to claim 9, wherein the biomolecules are chosen from one or more of the group consisting of protein, peptide, DNA, RNA, carbohydrates, lipids and combinations thereof.

11. A method for preparing cross-linked fragments from cross-linked biomolecules, biomolecular complexes or mixtures thereof as defined in claim 9, said method comprising the step of fragmenting the cross-linked biomolecules, biomolecular complexes or mixtures thereof.

12. A method for cleaving of an azide-reducing agent-sensitive scissile amide bond in a portion of cross-linked biomolecules, biomolecular complexes or mixtures thereof as defined in claim 35, or cross-linked fragments thereof, the carbonyl carbon atom of the amide bond being positioned 3 or 4 atoms from the azide group, and for reducing of the azide group to an amide group in another portion of the cross-linked biomolecules, biomolecular complexes or mixtures thereof, or cross-linked fragments thereof, said method comprising the steps of:
A) providing cross-linked biomolecules, biomolecular complexes or mixtures thereof, or cross-linked fragments thereof; and
B) subjecting the cross-linked biomolecules, biomolecular complexes or mixtures thereof, or cross-linked fragments thereof of step A) to an azide-reducing agent in a protic solvent thereby cleaving the cross-link in a portion of the cross-linked biomolecules, biomolecular complexes or mixtures thereof, or cross-linked fragments thereof, and reducing the azide group to an amine group in another portion of the cross-linked biomolecules, biomolecular complexes or mixtures thereof, or cross-linked fragments thereof.

13. The method according to claim 12, wherein the azide-reducing agent is chosen from the group, consisting of a H2/catalyst, tertiary phosphine, and a thiol-containing compound.

14. The method according to claim 13, wherein the azide-reducing agent is chosen from the group, consisting of a tertiary phosphine and a thiol-containing compound.

15. The method according to claim 14, wherein the tertiary phosphine is chosen from the group, consisting of tris(carboxyethyl)phosphine, tris(carboxypropyl)phosphine, tris(hydroxyethyl)phosphine, tris(hydroxypropyl)phosphine, tris(ethylamine)phosphine and tris(propylamine)phosphine.

16. The method according to claim 15, wherein the tertiary phosphine is tris(carboxyethyl)phosphine.

17. The method according to claim 13, wherein the thiol-containing compound is a dithiol-containing compound.

18. The method according to claim 17, wherein the dithiol-containing compound is dithiothreitol, butanedithiol or propanedithiol.

19. The method according to claim 12, wherein the azide-reducing agent is water-soluble.

20. The method according to claim 12, wherein step B) is carried out at a pH in the range of 3-10.

21. The method according to claim 12, wherein step B) is carried out at a pH in the range of 4-9.

22. A method for identifying cross-links in one or more cross-linked biomolecules, biomolecular complexes or mixtures thereof as defined in claim 35 or cross-linked fragments thereof, said method comprising the steps of:
a) providing said one or more cross-linked biomolecules, biomolecular complexes or mixtures thereof, and/or said cross-linked fragments;
b) optionally, fractionating said one or more cross-linked biomolecules, biomolecular complexes or mixtures thereof, and/or said cross-linked fragments into fractions comprising said one or more cross-linked biomolecules, biomolecular complexes or mixtures thereof, and/or said cross-linked fragments;
c) subjecting the cross-linked biomolecules, biomolecular complexes or mixtures thereof, and/or said cross-linked fragments of step a), or fractions thereof of step b) an azide-reducing agent in a protic solvent thereby cleaving the cross-link in a portion of the cross-linked biomolecules, biomolecular complexes or mixtures thereof, or cross-linked fragments thereof, and reducing the azide group to an amine group in another portion of the cross-linked biomolecules, biomolecular complexes or mixtures thereof, or cross-linked fragments thereof; and
d) identifying the cross-links by mass spectrometric analysis of the reaction mixture(s) of step c).

23. A method for identifying cross-links in a one or more cross-linked biomolecules, biomolecular complexes or mixtures thereof as defined in claim 35 and/or cross-linked fragments thereof, said method comprising the steps of:
I) providing said one or more cross-linked biomolecules, biomolecular complexes or mixtures thereof, and/or said cross-linked fragments;
II) fractionating said one or more cross-linked biomolecules, biomolecular complexes or mixtures thereof, and/or said cross-linked fragments into fractions;
III) to obtain reacted fractions subjecting the fractions of step II) to an azide-reducing agent in a protic solvent thereby cleaving the cross-link in a portion of the cross-linked biomolecules, biomolecular complexes or mixtures thereof, or cross-linked fragments thereof, and reducing the azide group to an amine group in another portion of the cross-linked biomolecules, biomolecular complexes or mixtures thereof, or cross-linked fragments thereof;
IV) fractionating said reacted fractions using the same fractionation technique used in step II) to separate reacted products from non-reacted products to obtain one or more reacted product fractions; and
V) identifying the cross-links by mass spectrometric analysis of the reacted product fractions of step IV).

24. The method according to claim 23, wherein the biomolecules are chosen from one or more of the group, consisting of protein, peptide, DNA, RNA, carbohydrates and lipids or combinations thereof.

25. The method according to claim 24, wherein the biomolecules are proteins.

26. The method according to claim 23, wherein step II) and/or step IV) are carried out by a chromatographic or electrophoretic fractionation technique.

27. The method according to claim 26, wherein step II) and/or step IV) are carried out by reversed phase chromatography.

28. A method for determining relative amounts of cross-links in a biomolecule or biomolecular complex in two or more samples, said method comprising the step of using at least a first cross-linker and a second cross-linker as defined in claim 1, said first and second cross-linker being identical in chemical composition and structure, and said first cross-linker or second cross-linker being enriched in one or more heavy isotopes resulting in a molar mass difference between said first and second cross-linker.

29. A method according to claim 28, said method comprising the steps of:
1) providing a first and second sample comprising one or more biomolecules, biomolecular complexes, or mixtures thereof;
2) preparing a first and a second cross-linked sample comprising one or more cross-linked biomolecules, biomolecular complexes, or mixtures thereof by cross-linking of said first sample with said first cross-linker, and of said second sample with said second cross-linker;
3) combining said first and second cross-linked sample to obtain a combined sample;
4) optionally, fragmenting said combined sample to obtain a fragmented combined sample;
5) performing on the combined sample of step 3) or the fragmented combined sample of step 4) the steps of
a) optionally, fractionating said one or more cross-linked biomolecules, biomolecular complexes or mixtures thereof, and/or said cross-linked fragments into fractions comprising said one or more cross-linked biomolecules, biomolecular complexes or mixtures thereof, and/or said cross-linked fragments;
b) subjecting the cross-linked biomolecules, biomolecular complexes or mixtures thereof, and/or said cross-linked fragments, or fractions thereof, to an azide-reducing agent in a protic solvent thereby cleaving the cross-link in a portion of the cross-linked biomolecules, biomolecular complexes or mixtures thereof, or cross-linked fragments thereof, and reducing the azide group to an amine group in another portion of the cross-linked biomolecules, biomolecular complexes or mixtures thereof, or cross-linked fragments thereof; and
c) identifying the cross-links by mass spectrometric analysis of the reaction mixture(s) of step b); and
6) determining the relative amount of each cross-link from the ratio of areas of the relevant peaks in mass spectra.

30. The method according to claim 22, wherein the biomolecules are chosen from one or more of the group, consisting of protein, peptide, DNA, RNA, carbohydrates and lipids or combinations thereof.

31. The method according to claim 30, wherein the biomolecules are proteins.

32. The method according to claim 22, wherein step b) is carried out by a chromatographic or electrophoretic fractionation technique.

33. The method according to claim 32, wherein step b) is carried out by reversed phase chromatography.

34. The method of claim 28, further comprising the steps of:
a) preparing a first and a second cross-linked sample comprising one or more cross-linked biomolecules, biomolecular complexes, or mixtures thereof by cross-linking of said first sample with said first cross-linker, and of said second sample with said second cross-linker;
b) combining said first and second cross-linked sample to obtain a combined sample;
c) optionally, fragmenting said combined sample to obtain a fragmented combined sample;
d) performing on the combined sample of step b) or the fragmented combined sample of step c) the steps of:
I) fractionating said one or more cross-linked biomolecules, biomolecular complexes or mixtures thereof, and/or said cross-linked fragments into fractions;
II) to obtain reacted fractions, subjecting the fractions of step I) to an azide-reducing agent in a protic solvent thereby cleaving the cross-link in a portion of the cross-linked biomolecules, biomolecular complexes or mixtures thereof, or cross-linked fragments thereof, and reducing the azide group to an amine group in another portion of the cross-linked biomolecules, biomolecular complexes or mixtures thereof, or cross-linked fragments thereof;
III) fractionating said reacted fractions using the same fractionation technique used in step I) to separate reacted products from non-reacted products to obtain one or more reacted product fractions; and
IV) identifying the cross-links by mass spectrometric analysis of the reacted product fractions of step III); and
e) determining the relative amount of each cross-link from the ratio of areas of the relevant peaks in mass spectra.

35. A cross-linked biomolecule, a biomolecular complex, or a mixture thereof comprising a cross-linker according to claim 1.
